(19) United States
(12) Patent Application Publication
(10) Pub. No.: US 2004/0071801 A1
(43) Pub. Date: Apr. 15, 2004

(54) Herbal formulation of Gymnema sylvestre as a dietary aid

(76) Inventors: Drew Edell, Upper Montclair, NJ (US); Richard A. Handel, West Calowell, NJ (US)

Correspondence Address: Marguerite Del Valle, Trademark and Patent Counselors of America, P. C., 19th Floor, 915 Broadway, New York, NY, 10010, US

(21) Appl. No.: 10/268,331

(22) Filed: Oct. 9, 2002

(51) Int. Cl. A61K 35/78

(52) U.S. Cl. 424/762; 424/756

(57) ABSTRACT

The present invention incorporates gymnemic acid, derived from Gymnema sylvestre, with a debittering agent into a slow-dissolving lozenge, along with other compounds which affect the taste for sweetness, to provide a dietary aid for persons having difficulty controlling their sweet intake.

### FIELD OF THE INVENTION

The present invention relates to a herbal formulation that is useful as a dietary aid in controlling caloric intake derived from sweets. Gymnemic acid, derived from the herb Gymnema sylvestre, is known to block sweet receptors on the tongue. Gymnema sylvestre, however, has a bitter and unpleasant taste, making it difficult to promote as a dietary supplement. The present invention incorporates gymnemic acid, derived from Gymnema sylvestre, with a debittering agent into a slow-dissolving lozenge, along with other compounds which affect the taste for sweetness, to provide a dietary aid for persons having difficulty controlling their sweet intake.

### BACKGROUND OF THE INVENTION

Gymnema sylvestre is a woody, climbing plant, native to India, Africa and China. It has been used in the treatment of diabetes, impaired glucose tolerance, and various conditions associated with diabetes. See, U.S. Pat. No. 5,980,902 to Shanmugasundaram et al. U.S. Pat. No. 5,900,240 to Tomer et al, discloses the use of Gymnema sylvestre as a dietary supplement useful for lowering the glucose level of the blood in those suffering from diabetes mellitus. See also, U.S. Pat. No. 5,730,988 to Womack. U.S. Pat. No. 4,761,286 to Hiji teaches the use of Gymnema sylvestre to inhibit the absorption of glucose in the intestinal tract. Gymnema sylvestre has also been incorporated into dietary supplements which are purported to reduce the build-up of body fat. See, U.S. Pat. No. 5,612,039 to Policappelli et al. It also provides a cariostatic effect by preventing the formation of plaque on tooth surfaces. U.S. Pat. No. 4,912,089 to Hiji.

It has long been recognized that chewing the leaves of Gymnema sylvestre destroys the ability to taste the sweetness of foods consumed thereafter for a period of several hours. This property gives the herb its common name, “gurmar” or sugar destroyer. This effect has long been known, for example, Gymnema sylvestre was introduced to a British officer, Captain Edgeworth, and his wife more than a century ago by the inhabitants of an Indian village. Upon chewing the leaves, the couple found that the sweetness of their tea vanished. “Biological effects of Gymnema sylvestre fractions,” Yackzan, K. S. Ala. J. Med. Sci. 3:1-9 (1966).

Studies over the years have isolated the active compound, gymnemic acid, that causes the suppression of taste sensitivity to sweet substances. “Antisweet Activity of Gymnemic Acid Al and Its Derivatives,” Kurihara, Y, Life Sciences 8(1):537 (1969); “Gymnemic Acid, the Antisaccharine Principle of Gymnema sylvestre,” Dateo et al, J. Agr. Food Chem. 21 (5):899 (1973). In order to assay the antisweet activity of Gymnema sylvestre, investigators have administered oral rinses of gymnemic acid solutions to subjects. Kurihara at 539; Dateo at 900. A survey of various studies utilizing oral rinses of gymnemic acid is presented in “Effects of gymnema sylvestre and synsepalum dulcificum on taste in man,” Bartoshuck, L. M. et al, Psychophysics and Sensory Coding, pp. 436-437.

U.S. Pat. No. 5,605,698 to Ueno discloses various oral compositions incorporating gymnemic acid as a dietary aid in the form of a chewable gum, chewable tablet, oral refrigerant, or oral troche. None of these compositions, however, incorporate the debittering agents or sweet affectors of the present invention.

The formulations of the present invention incorporate gymnemic acid into a neutral or pleasant tasting lozenge that dissolves slowly, evenly distributing the active ingredient throughout the mouth. The gymnemic acid, having a similar molecular configuration to glucose, occupies and therefore blocks the sweet receptors. A person who dissolves one of the lozenges in his or her mouth therefore will not be able to taste or enjoy sweet foods. Sweet foods, in fact, will have an unpleasant taste which, in time, will cause an aversion in the steady user to sweet foods. By setting up such an aversion therapy process, the steady user of the lozenges of the present invention will cease to crave sweet foods and will develop eating habits that avoid sweet foods. He or she will thus be better able to lower his or her caloric intake without the feelings of deprivation that often sabotage weight loss programs.

### SUMMARY OF THE INVENTION

It has been discovered according to the present invention that a combination of certain compounds, including Gymenma sylvestre, can be incorporated into a lozenge form which, when dissolved in a the mouth of the user, destroys the craving for sweets and enables that person to gain control over his or her caloric intake and thereby succeed in a weight loss program.

The lozenge of the present invention incorporates Gymnema sylvestre and a debittering agent. When taken regularly before meals or when sweet foods will be available, a user will develop an aversion to sweets which will enable him or her to reduce caloric intake derived from sweet foods.

Accordingly, the present invention provides a novel herbal formulation useful as a dietary aid in controlling caloric intake by delivering gymnemic acid in a slow dissolving lozenge form to the sweet receptors in the mouth.

### DETAILED DESCRIPTION OF THE INVENTION

Lozenges incorporating gymnemic acid and a debittering agent are prepared. Suitable debittering agents include but are not limited to L-lysine, maltol, ethylmaltol, glycyrrhizin thaumatin, and soy lysophospholipids. Other compounds, such as ziziphus jujuba or horenias dulcis, which affect the ability to taste the sweetness of foods may also be added.

Using conventional procedures, lozenges of the following composition are prepared:

| Ingredient | milligrams per tablet |
|---|---|
| Sorbitol, directly compressible | 458.0000 |
| Stearic acid, kosher, vegetable-source | 9.1000 |
| Bitter masking agent, natural (Comax | 3.5000 |
| Flavors #2588E17379) | |
| Peppermint flavor (Flavors of North | 1.6000 |
| America #894.043) | |
| Magnesium stearate, kosher, vegetable- | 1.3000 |
| source | |
| Gymnema sylvestre leaf extract, 75% | 1.5000 |
| gymnemic acid | |
| TOTAL | 475.0000 |

The user lets one lozenge dissolve in the mouth between meals or whenever the user feels an urge to eat or drink high-calorie sweets or beverages containing sugar. The lozenge thus prepared and used neutralizes the taste of sweetness for several hours, and curbs sweetness cravings such that the user can avoid ingesting sweet foods and between-meal snacks, without feelings of deprivation. The user may then establish healthy and low-calorie eating habits without having to battle the difficulties arising from sweetness cravings.

1. An herbal formulation useful as a dietary aid for controlling caloric intake comprising gymnemic acid and a debittering agent incorporated into a lozenge form.

2. An herbal formulation according to claim 1 wherein the debittering agent is L-lysine.

3. An herbal formulation according to claim 1 wherein the debittering agent is maltol.

4. An herbal formulation according to claim 1 wherein the debittering agent is ethylmaltol.

5. An herbal formulation according to claim 1 wherein the debittering agent is glycyrrhizin thaumatin.

6. An herbal formulation according to claim 1 wherein the debittering agent is a soy lysophospholipid.

7. An herbal formulation useful as a dietary aid for controlling caloric intake comprising gymnemic acid, a debittering agent, and Ziziphus jujuba incorporated into a lozenge form.

8. An herbal formulation useful as a dietary aid for controlling caloric intake comprising gymnemic acid, a debittering agent, and Horenias dulcis incorporated into a lozenge form.
